(19) United States
(12) Patent Application Publication
(10) Pub. No.: US 2025/0152174 A1
(43) Pub. Date: May 15, 2025

(54) INTRAVASCULAR INDWELLING MEDICAL DEVICE

(71) Applicants: SB-KAWASUMI LABORATORIES, INC., Kawasaki-shi, Kanagawa (JP); SUMITOMO BAKELITE CO., LTD., Tokyo (JP)

(72) Inventors: Kenjiro Yamaguchi, Kawasaki-shi (JP); Yasuhiro Fujita, Kawasaki-shi (JP)

(73) Assignees: SB-KAWASUMI LABORATORIES, INC., Kawasaki-shi, Kanagawa (JP); SUMITOMO BAKELITE CO., LTD., Tokyo (JP)

(21) Appl. No.: 18/837,944

(22) PCT Filed: Mar. 22, 2023
(86) PCT No.: PCT/JP2023/011213

§ 371 Date: Aug. 13, 2024

(30) Foreign Application Priority Data
Mar. 31, 2022 (JP) 2022-058439
Mar. 9, 2023 (JP) 2023-036540

(51) Int. Cl. A61B 17/12; A61B 17/00; A61B 90/00

(52) U.S. Cl. CPC A61B 17/12109; A61B 17/12145; A61B 17/12172; A61B 17/12177; A61B 17/1219; A61B 90/39; A61B 2017/00477; A61B 2017/00898; A61B 2017/00942; A61B 2017/1205

(57) ABSTRACT

The present invention provides an intravascular indwelling medical device having a structure capable of occluding a desired site within a blood vessel in a shorter time. An intravascular indwelling medical device (100) of the present invention is an intravascular indwelling medical device that dwells within a blood vessel (360) to occlude the blood vessel (360). The intravascular indwelling medical device (100) includes a mesh-form main body part (10) and a water-absorbing swelling material (20) that is disposed within the main body part (10) and swells upon absorbing water.

### FIELD OF THE INVENTION

The present invention relates to an intravascular indwelling medical device.

### BACKGROUND OF THE INVENTION

Examples of an intravascular indwelling medical device that dwells within a blood vessel and occludes the blood vessel include an intravascular indwelling medical device described in Patent Literature 1.

The intravascular indwelling medical device of Patent Literature 1 (described as an occlusion device in the same literature) includes a plurality of mesh-form main body cages (described as a mesh carriage in the same literature) and a connection member (described as a marker in the same literature) to which a pusher wire (described as a core wire in the same literature) is connected.

### DESCRIPTION OF THE RELATED ART

### Patent Literature

Patent Literature 1: Japanese Unexamined Patent Application, First Publication No. 2021-120028

### SUMMARY OF THE INVENTION

### Problems to be Solved

According to the study by the present inventors, there is room for improvement in the intravascular indwelling medical device described in PTL 1 from the viewpoint of occluding a desired site within a blood vessel in a short time.

The present invention has been made in view of the above-described problems, and an object of the present invention is to provide an intravascular indwelling medical device having a structure that makes it possible to occlude a desired site within a blood vessel in a shorter time.

### Solution to Solve the Problems

According to the present invention, there is provided an intravascular indwelling medical device that dwells within a blood vessel to occlude the blood vessel, the intravascular indwelling medical device including a mesh-form main body part, and a water-absorbing swelling material that is disposed within the main body part and swells upon absorbing water.

### Effects of the Invention

According to the present invention, a desired site within a blood vessel can be occluded in a shorter time.

### BRIEF DESCRIPTION OF THE DRAWINGS

FIG. 1 A side view of an intravascular indwelling medical device according to an embodiment, which shows a state in which the intravascular indwelling medical device is accommodated in a guide catheter.

FIG. 2A A side view of the intravascular indwelling medical device according to the embodiment, which shows a state before a water-absorbing swelling material swells.

FIG. 2B A side view of the intravascular indwelling medical device according to the embodiment, which shows a state in which the water-absorbing swelling material is swelled.

FIG. 3A A cross-sectional view taken along line A-A shown in FIG. 2A.

FIG. 3B A cross-sectional view taken along line B-B shown in FIG. 2B.

FIG. 4A A side view for describing a series of operations in a case where the intravascular indwelling medical device dwells within a blood vessel, which shows a state in which the intravascular indwelling medical device is accommodated in the guide catheter.

FIG. 4B A side view for describing a series of operations in a case where the intravascular indwelling medical device dwells within the blood vessel, which shows a state in which the intravascular indwelling medical device is released from restraint by the guide catheter.

FIG. 5A A side view of the intravascular indwelling medical device in the embodiment, which shows a state in which the intravascular indwelling medical device dwells within the blood vessel.

FIG. 5B A side view of the intravascular indwelling medical device in the embodiment, which shows a state in which the intravascular indwelling medical device dwells within the blood vessel.

FIG. 5C A side view of the intravascular indwelling medical device in the embodiment, which shows a state in which the intravascular indwelling medical device dwells within the blood vessel.

FIG. 6A A cross-sectional view taken along line A-A shown in FIG. 5A.

FIG. 6B A cross-sectional view taken along line B-B shown in FIG. 5C.

FIG. 7 A diagram showing each of a first region and a second region in a cross section perpendicular to an axial direction of the intravascular indwelling medical device according to the embodiment.

FIG. 8 A diagram showing each of a third region and a fourth region in the cross section perpendicular to the axial direction of the intravascular indwelling medical device according to the embodiment.

FIG. 9A A side view of an intravascular indwelling medical device according to a modification example of the embodiment, which shows a state before the water-absorbing swelling material swells.

FIG. 9B A side view of the intravascular indwelling medical device according to the modification example of the embodiment, which shows a state in which the water-absorbing swelling material is swelled.

FIG. 10A A cross-sectional view taken along line A-A shown in FIG. 9A.

FIG. 10B is a cross-sectional view taken along line B-B shown in FIG. 9B.

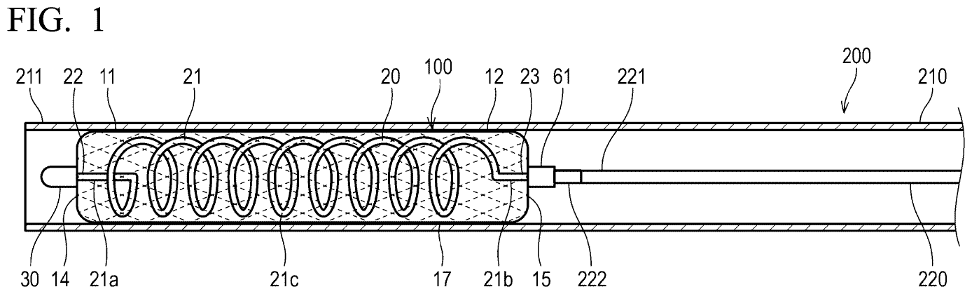

Hereinafter, embodiments of the present invention will be described with reference to FIGS. 1 to 8. In all the drawings, the same components are denoted by the same reference signs, and the description thereof will be omitted as appropriate. In FIGS. 1, 2A, 2B, and 4A to 5C, stitches of a main body part 10 are indicated by a broken line for convenience in order to make an internal structure of the main body part 10 easy to see.

In addition, various components of an intravascular indwelling medical device 100 according to the embodiment of the present invention do not need to be independent of each other, and it is allowed that one component is a part of another component, a part of a certain component and a part of another component overlap each other, and the like.

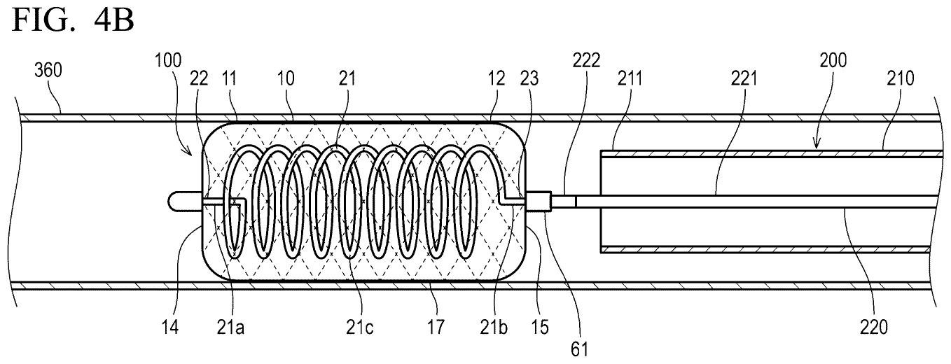

As shown in FIG. 4A, FIG. 4B, and the like, the intravascular indwelling medical device 100 according to the present embodiment is an intravascular indwelling medical device that dwells within a blood vessel 360 to occlude the blood vessel 360.

The intravascular indwelling medical device 100 includes a mesh-form main body part 10 and a water-absorbing swelling material 20 that is disposed within the main body part 10 and swells upon absorbing water.

The intravascular indwelling medical device 100 is transported to a desired site within the blood vessel 360 in a state of being accommodated in a guide catheter 210 which will be described below.

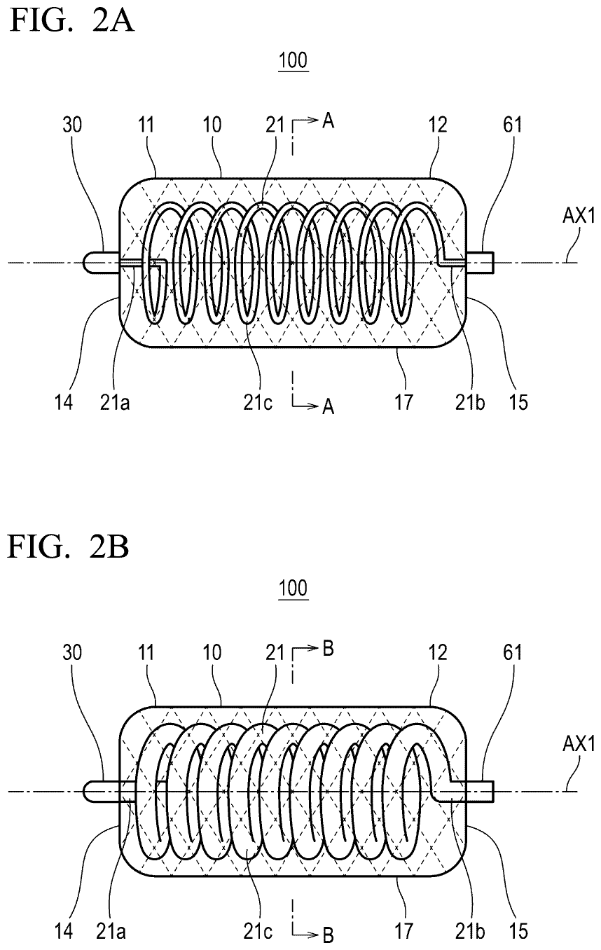

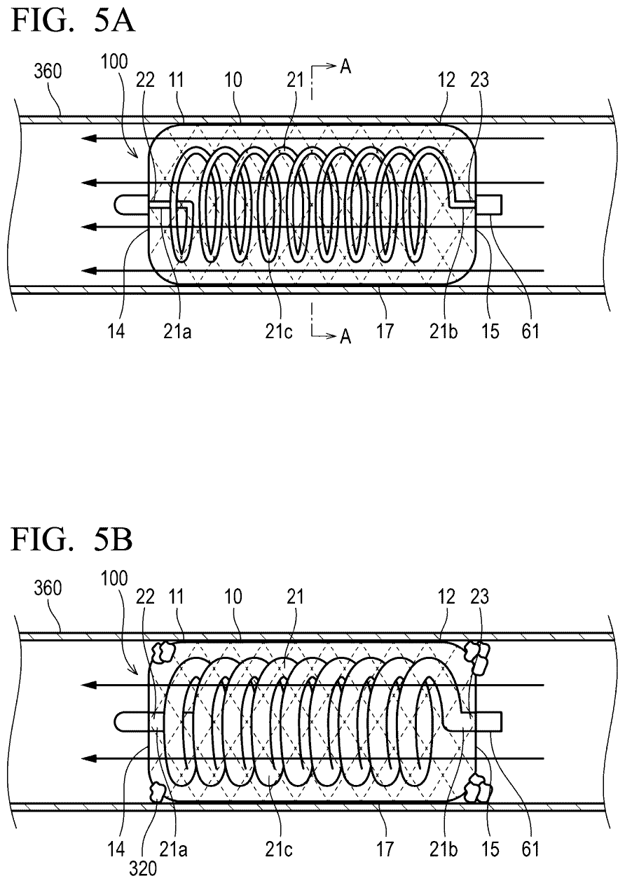

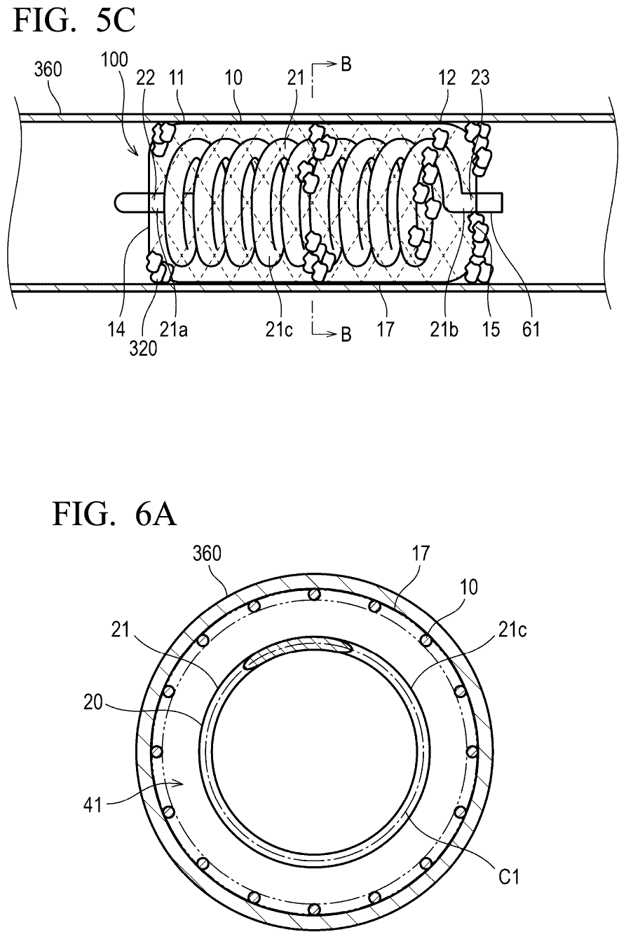

An outer diameter of the intravascular indwelling medical device 100 is set to a dimension larger than an inner diameter of the guide catheter 210. Therefore, while being accommodated in the guide catheter 210, the intravascular indwelling medical device 100 is in a reduced diameter state in which the outer diameter and an inner diameter of the intravascular indwelling medical device 100 are relatively small (see FIG. 1). Then, in a case where the intravascular indwelling medical device 100 is released from restraint by the guide catheter 210, the intravascular indwelling medical device 100 elastically restores from the reduced diameter state to an expanded diameter state (see FIG. 2A). As a result, the intravascular indwelling medical device 100 dwells (is fixed) at a desired site within the blood vessel 360. Then, the desired sire of the blood vessel 360 is occluded by the intravascular indwelling medical device 100 that is in the expanded diameter state and thrombi formed inside and around the intravascular indwelling medical device 100 (see FIGS. 5A, 5B, and 5C). Further, after the dwelling of the intravascular indwelling medical device 100, a periphery of the intravascular indwelling medical device 100 on an inner wall of the blood vessel 360 is organized, so that the intravascular indwelling medical device 100 is more firmly fixed to a desired site within the blood vessel 360.

The reduced diameter state of the intravascular indwelling medical device 100 means a state in which the intravascular indwelling medical device 100 is compressed in a radial direction to an extent that the intravascular indwelling medical device 100 can be present within the guide catheter 210. In addition, the expanded diameter state of the intravascular indwelling medical device 100 means a state in which the intravascular indwelling medical device 100 is expanded in diameter at least more than the reduced diameter state and is, for example, a natural state of the intravascular indwelling medical device 100.

In the following descriptions, a shape of each part of the intravascular indwelling medical device 100 will be described based on a shape in a natural state in which no external force is applied, unless otherwise specified.

According to the present embodiment, the intravascular indwelling medical device 100 further includes the water-absorbing swelling material 20 that is disposed within the main body part 10 and swells by absorbing water, in addition to the mesh-form main body part 10.

As a result, the blood vessel 360 can also be occluded by the water-absorbing swelling material 20 that swells upon absorbing water, in addition to the thrombi formed inside and around the intravascular indwelling medical device 100. Therefore, the desired site within the blood vessel 360 can be occluded in a short time as compared with a case where the intravascular indwelling medical device 100 does not include the water-absorbing swelling material 20.

In addition, since the mesh-form main body part 10 is locked to the inner wall of the blood vessel 360, it is possible to suppress displacement of the intravascular indwelling medical device 100 relative to the blood vessel 360 by a pressure of a blood flow. That is, since the intravascular indwelling medical device 100 can maintain a state in which the intravascular indwelling medical device 100 dwells in a desired site within the blood vessel 360, the desired site can be more reliably occluded.

As described above, according to the present embodiment, the desired site within the blood vessel 360 can be more reliably occluded in a shorter time.

The intravascular indwelling medical device 100 is transported to and dwells in the desired site within the blood vessel 360 by a delivery device 200 (see FIG. 1). In FIGS. 1, 4A, and 4B, the guide catheter 210 is shown in a cross-sectional view taken along an axial direction of the guide catheter 210.

The delivery device 200 includes, for example, the guide catheter 210 described above in which the intravascular indwelling medical device 100 is accommodated, and a pusher wire 220 that is connected to the intravascular indwelling medical device 100.

The guide catheter 210 is formed, for example, in a tubular shape that is long in one direction. An operating part (not shown) is provided at a proximal end part of the guide catheter 210.

The guide catheter 210 is externally mounted to the intravascular indwelling medical device 100 to be removable by being slid in an axial direction relative to the intravascular indwelling medical device 100.

While being accommodated in the guide catheter 210, the intravascular indwelling medical device 100 is restrained by the guide catheter 210 on an outer periphery thereof and is compressed in the radial direction to the extent that can be present within the guide catheter 210.

The pusher wire 220 is composed of, for example, a wire member that is long in one direction. A distal end part 221 of the pusher wire 220 is provided with, for example, a mounting part 222 that is detachably mounted to a connection member 61 described below by screwing. In the present embodiment, a screw thread is formed on an outer peripheral surface of the mounting part 222, and the mounting part 222 has a male screw shape. The distal end part 221 of the pusher wire 220 is connected to a proximal end part 12 of the main body part 10 via the mounting part 222. The pusher wire 220 to which the intravascular indwelling medical device 100 is connected is inserted into the guide catheter 210 and pushed from a proximal end side to a distal end side, whereby the intravascular indwelling medical device 100 can be transported to a desired site in the guide catheter 210.

A material of the guide catheter 210 is not particularly limited, but is preferably a fluororesin having a low friction resistance, such as polytetrafluoroethylene. As a result, it is possible to reduce a sliding resistance of the guide catheter 210 with respect to the inner wall of the blood vessel 360 in a case where the guide catheter 210 moves within a biological organ.

A material of the pusher wire 220 is not particularly limited, and examples thereof include a superelastic alloy such as nitinol.

For example, it is also preferable that an outer peripheral surface of the guide catheter 210 is coated with a hydrophilic agent. As a result, it is possible to reduce the sliding resistance of the guide catheter 210 with respect to the inner wall of the blood vessel 360, and the operability of the guide catheter 210 is improved.

As shown in FIG. 1, in the present embodiment, the main body part 10 is formed, for example, in a hollow cylindrical shape. More specifically, the entire main body part 10 is braided in a hollow cylindrical shape by a plurality of wire members, for example.

A material of the wire member constituting the main body part 10 is not particularly limited, and examples thereof include a superelastic alloy such as nitinol. Note that the material of the wire member constituting the main body part 10 may be, for example, a resin material.

Each of an outer diameter and an inner diameter of the main body part 10 is, for example, substantially constant regardless of a position of the main body part 10 in an axial direction. However, in the present invention, each of the inner diameter and the outer diameter of the main body part 10 may be changed in the axial direction of the main body part 10. More specifically, the main body pan 10 may include, for example, a plurality of large-diameter parts having a relatively large outer diameter and inner diameter, and a plurality of constricted parts (small-diameter parts) having a relatively small outer diameter and inner diameter. In this case, in the axial direction of the main body part 10, one large-diameter part and one constricted part are alternately disposed in order from the distal end side. In the axial direction of the main body part 10, the large-diameter parts are arranged to be spaced apart from each other.

In the following description, a circumferential direction of the main body part 10 may be simply referred to as a circumferential direction, the axial direction of the main body part 10 may be simply referred to as an axial direction, and a radial direction of the main body part 10 may be simply referred to as a radial direction. In addition, a distal end-proximal end direction is a longitudinal direction of the main body part 10. An axis means a central axis along the longitudinal direction of the main body part 10.

As described above, in a case where the intravascular indwelling medical device 100 is transported to the desired site within the blood vessel 360, the intravascular indwelling medical device 100 is accommodated in the guide catheter 210. In this state, the main body part 10 is restrained by the guide catheter 210 and is compressed in the radial direction to the extent that can be present within the guide catheter 210 (in the reduced diameter state) (see FIGS. 1 and 4A). Further, the main body part 10 is expanded outward in the axial direction. Then, in a case where the intravascular indwelling medical device 100 is released from the restraint by the guide catheter 210, the main body pan 10 is elastically restored to a natural state (see FIG. 41) by an elastic restoring force of the wire member constituting the main body part 10 and an elastic restoring force of the water-absorbing swelling material 20 having a coil shape which will be described below. More specifically, in a case where the main body part 10 is elastically restored from the reduced diameter state to the natural state, the main body part 10 expands in diameter outward in the radial direction and contracts inward in the axial direction.

As shown in FIGS. 2A and 2B, the water-absorbing swelling material 20 is, for example, fixed to a distal end part 11 and the proximal end part 12 of the main body part 10.

As a result, the water-absorbing swelling material 20 can favorably follow the expansion and contraction of the main body part 10 in the axial direction.

More specifically, as shown in FIG. 2A and FIG. 2B, the water-absorbing swelling material 20 is formed in, for example, a shape in which a wire 21 is wound in a coil shape.

One end 22 of the wire 21 is fixed to the distal end part 11 of the main body part 10, and the other end 23 of the wire 21 is fixed to the proximal end part 12 of the main body part 10.

As a result, the water-absorbing swelling material 20 can be favorably expanded and contracted in an axis direction of the wire 21 having a coil shape. Therefore, the water-absorbing swelling material 20 can favorably follow the expansion and contraction of the main body part 10 in the axial direction.

In addition, in a case where the intravascular indwelling medical device 100 is released from the restraint by the guide catheter 210, the water-absorbing swelling material 20 can be rapidly expanded in diameter outward in the radial direction.

Further, in the water-absorbing swelling material 20, since a contact area that comes in to contact with the blood flow can be sufficiently ensured, the water-absorbing swelling material 20 can rapidly absorb a moisture in the blood and can sufficiently swell. Therefore, the desired site within the blood vessel 360 can be more reliably occluded in a shorter time.

More specifically, the wire 21 includes, for example, a distal end pan 21a, a proximal end part 21b, and an intermediate part 21c that is located between the distal end part 21a and the proximal end pan 21b. A distal end of the distal end pan 21a is fixed to the distal end part 11 of the main body part 10, and a proximal end of the proximal end part 21b is fixed to the proximal end part 12 of the main body part 10.

Each of the distal end part 21a and the proximal end part 21b extends linearly along an axis AX1 (see FIGS. 2A and 2B) of the water-absorbing swelling material 20 having a coil shape.

The intermediate part 21c is wound in a spiral shape and has a plurality of winding parts. Each winding part is a part formed by winding the wire 21 around one turn. It is preferable that inner diameters of the winding parts in the intermediate part 21c are equal to each other. Similarly, it is preferable that outer diameters of the winding parts in the intermediate part 21c are equal to each other. In addition, the plurality of winding parts are disposed at equal intervals as an example.

The intermediate part 21c is disposed coaxially with the main body part 10. That is, the water-absorbing swelling material 20 is disposed coaxially with the main body part 10.

An outer diameter of the intermediate part 21c is set to, for example, a dimension smaller than the inner diameter of the main body part 10, and a gap 41 is formed between an outer peripheral edge of the intermediate part 21c (an outer peripheral edge of the water-absorbing swelling material 20) and an inner peripheral surface of the main body part 10. As shown in FIG. 5A, immediately after the intravascular indwelling medical device 100 dwells, the blood flow can pass through the gap 41 or the inside of the intermediate part 21c.

In the present embodiment, the wire 21 is formed of, for example, a water-absorbent polymer.

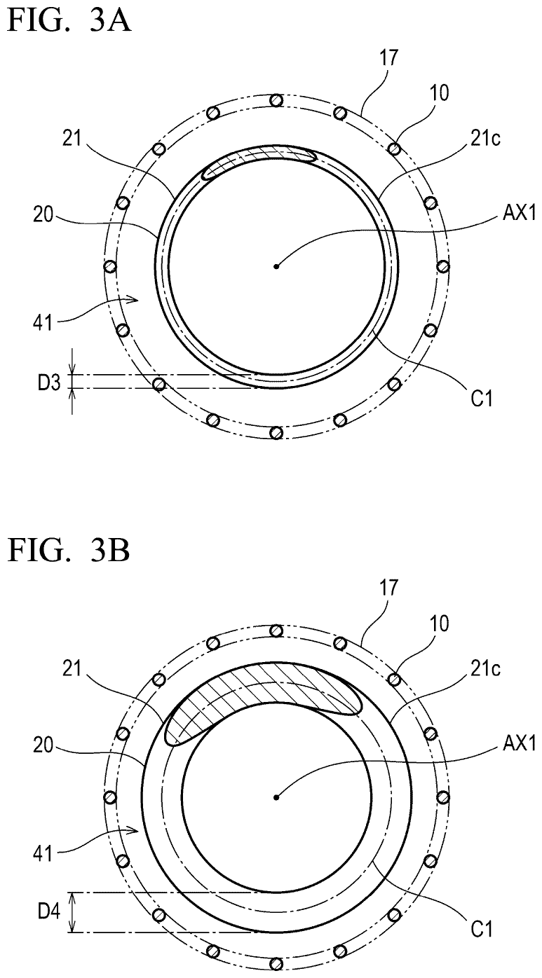

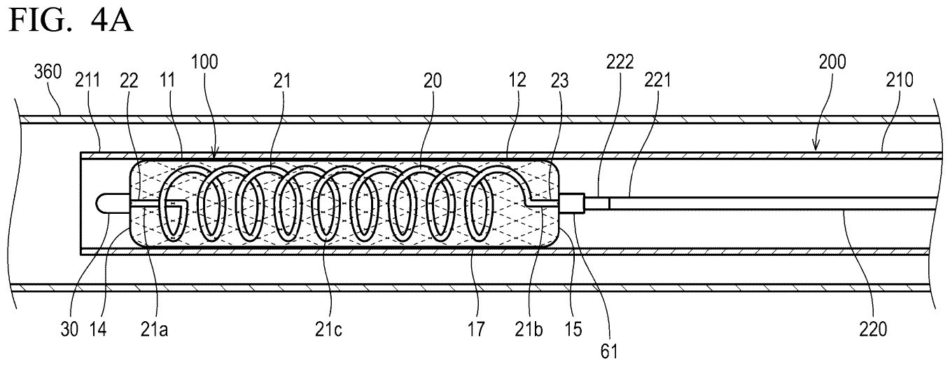

As a result, as shown in FIGS. 3A and 3B, the wire 21 absorbs the moisture in the blood so that a wire diameter of the water-absorbing swelling material 20 increases. More specifically, a thickness of the water-absorbing swelling material 20 increases substantially uniformly outward in the radial direction of the wire 21 with respect to a center line C1 of the wire 21. As a result, an inner diameter of the water-absorbing swelling material 20 after swelling and the gap 41 between the outer peripheral edge of the water-absorbing swelling material 20 after swelling and the inner peripheral surface of the main body part 10 can be made small. Therefore, the growth of the thrombi is promoted in an inner cavity of the water-absorbing swelling material 20 and the gap 41.

In the present embodiment, a wire diameter (D4 shown in FIG. 38) of the water-absorbing swelling material 20 that is fully swelled is, for example, preferably 1.5 times or more of a wire diameter (D3 shown in FIG. 3A) of the water-absorbing swelling material 20 before swelling, and more preferably 2 times or more of the wire diameter D3.

Examples of water-absorbent polymers that can be used include polyacrylic acid (such as a copolymer of acrylic acid and vinyl alcohol, or a polymer of sodium acrylate), polymethacrylic acid, polyacrylamide, polyhydroxyethyl methacrylate and a derivative thereof, a crosslinked polymer of a polyol such as polyvinyl alcohol, polyvinylpyrrolidone, and polyethylene glycol, and a polysaccharide-based hydrogel (such as an alkaline hydrolysate of a graft copolymer of starch and acrylonitrile).

Here, in the present embodiment, as shown in FIG. 1. FIG. 2A, and the like, the connection member 61 for connecting the intravascular indwelling medical device 100 to the pusher wire 220 is provided at the proximal end part 12 of the main body part 10, and a marker member 30 is provided at the distal end part 11 of the main body part 10.

The water-absorbing swelling material 20 is fixed to the connection member 61, and thus is fixed to the proximal end part 12 of the main body part 10.

With such a configuration, the water-absorbing swelling material 20 is not directly fixed to the proximal end part 12 of the mesh-form main body part 10 but is fixed via the connection member 61 provided at the proximal end part 12. Therefore, the water-absorbing swelling material 20 can be favorably fixed to the proximal end part 12 of the main body part 10.

Similarly, the water-absorbing swelling material 20 is fixed to the distal end part 11 of the main body part 10 by being fixed to the marker member 30.

With such a configuration, the water-absorbing swelling material 20 is not directly fixed to the distal end part 11 of the mesh-form main body part 10 but is fixed via the marker member 30 provided at the distal end part 11. Therefore, the water-absorbing swelling material 20 can be favorably fixed to the distal end part 11 of the main body part 10.

In addition, in the present embodiment, each of the connection member 61 and the marker member 30 is coaxially disposed with the main body part 10. Then, in a case where the main body part 10 expands in the axial direction, the connection member 61 and the marker member 30 move outward in the axial direction (a direction in which the connection member 61 and the marker member 30 move away from each other in the axial direction) while maintaining a state of being coaxially disposed with the main body part 10. Similarly, in a case where the main body part 10 contracts in the axial direction, the connection member 61 and the marker member 30 move inward in the axial direction (a direction in which the connection member 61 and the marker member 30 approach each other in the axial direction) while maintaining a state of being coaxially disposed with the main body part 10.

Therefore, as described above, since the water-absorbing swelling material 20 is favorably fixed to the distal end part 11 and the proximal end part 12 of the main body part 10 via the connection member 61 and the marker member 30, the water-absorbing swelling material 20 can favorably follow the expansion and contraction of the main body part 10 in the axial direction while maintaining a state of being disposed coaxially with the main body part 10.

More specifically, in the wire 21, the proximal end part 21b (the other end 23 of the wire 21) is fixed to the connection member 61, and the distal end part 21a (the one end 22 of the wire 21) is fixed to the marker member 30. Then, the intermediate part 21c, which is a portion located between the distal end part 21a and the proximal end part 21b in the wire 21 and has the plurality of winding parts, follows the main body part 10 to favorably expand and contract in the axial direction while maintaining a state of being coaxially disposed with the main body part 10.

As shown in FIGS. 2A and 2B, the connection member 61 is disposed, for example, at a center (axis center) of the proximal end part 12 of the main body part 10. The connection member 61 protrudes, for example, from a proximal end surface 15 of the main body part 10 toward the proximal end side. The connection member 61 is disposed, for example, at a central part of the proximal end surface 15 of the main body part 10. The connection member 61 is formed in, for example, a substantially cylindrical shape and is disposed coaxially with the main body part 10 as described above.

The connection member 61 has, for example, a female screw shape that is detachably screwed to the mounting part 222 of the pusher wire 220. The connection member 61 is screwed to the mounting part 222, so that the main body part 10 is connected to the distal end part 221 of the pusher wire 220. That is, the intravascular indwelling medical device 100 and the pusher wire 220 are connected to each other via the connection member 61. In addition, by releasing the screwing between the connection member 61 and the mounting part 222, the connection between the intravascular indwelling medical device 100 and the pusher wire 220 can be released.

As shown in FIGS. 2A and 2B, the marker member 30 protrudes, for example, from a distal end surface 14 of the main body part 10 toward the distal end side. The marker member 30 is disposed, for example, at a central part of the distal end surface 14 of the main body part 10. The marker member 30 is formed in, for example, a cylindrical shape and is coaxially disposed with the main body part 10 as described above.

The marker member 30 is made of, for example, a radiopaque metallic material. In the blood vessel 360, a position of the distal end part 11 of the main body part 10 can be recognized by the marker member 30.

Here, a preferred disposition of the water-absorbing swelling material 20 in a cross section perpendicular to the axial direction will be described with reference to FIG. 7. In FIG. 7, a first region R1 described below is indicated by hatching upward to the left, and a second region R2 described below is indicated by hatching upward to the right. In addition, in FIG. 7, the wire member of the main body part 10 is indicated by a broken line. FIG. 7 shows a cross section taken along line A-A of FIG. 2A.

In order to ensure that an entire area of an internal space of the main body pan 10 is filled in a well-balanced manner by the swelling of the water-absorbing swelling material 20 having a coil shape, it is preferable that, in the internal space of the main body part 10, a volume of a space inside the water-absorbing swelling material 20 having a coil shape (first region R1 shown in FIG. 7; more precisely, an inner space with respect to the center line C1 described later) and a volume of a space outside the water-absorbing swelling material 20 (second region R2 shown in FIG. 7; more precisely, an outer space with respect to the center line C1 described later) are substantially equal to each other.

In a case where the outer diameter of the main body part 10 in a natural state is denoted by D and a distance from the axis AX1 of the water-absorbing swelling material 20 having a coil shape to the center line C1 extending in a spiral shape of the wire 21 constituting the water-absorbing swelling material 20 is denoted by L, L such that a cross-sectional area of the first region R1 and a cross-sectional area of the second region R2 are equal to each other is L=(⅛^{1/2})·D≈0.35D.

Regarding an upper limit and a lower limit of L interposing such L, in a case where a value of L at which a ratio of the cross-sectional area of the second region R2 to the cross-sectional area of the first region R1=2:1 is set as a lower limit value of L, and a value of L at which the cross-sectional area of the second region R2 to the cross-sectional area of the first region R1=1:2 is set as an upper limit value of L, ( 1/12^{1/2})·D≤L≤(⅙^{1/2})·D is established, and thus, approximately 0.29D≤L≤0.41D is established.

In a case where the lower limit of L is set to 0.25D, which is slightly smaller than 0.29D, 0.25D≤L≤0.41D is established.

That is, as shown in FIG. 7, in a case where the outer diameter of the main body part 10 in a natural state is denoted by D and a distance from the axis AX1 of the water-absorbing swelling material 20 having a coil shape to the center line C1 extending in a spiral shape of the wire 21 is denoted by L, it is preferable that 0.25D<L<0.41D is satisfied. In FIGS. 3A, 3B, 6A, 6B, 7, and 8, the center line C1 is indicated by a one-dot chain line, and each of the inner peripheral surface and an outer peripheral surface of the main body part 10 is indicated by a two-dot chain line.

With such a configuration, in a cross section perpendicular to the axial direction (cross section along the radial direction) (FIG. 7), the first region R1 and the second region R2 can be filled with the swelled water-absorbing swelling material 20 in a well-balanced manner. Therefore, thrombi 320 can grow in a well-balanced manner in each of the first region R1 and the second region R2 in the internal space of the main body part 10.

As described above, in a case where the value of L at which a ratio of the cross-sectional area of the second region R2 to the cross-sectional area of the first region R1 is 2:1 is set as the lower limit value of L, since 0.29D≤L is established, 0.29D≤L is preferable.

Further, a more preferable disposition of the water-absorbing swelling material 20 in a cross section perpendicular to the axial direction will be described.

In a case where a value of L at which a ratio of the cross-sectional area of the second region R2 to the cross-sectional area of the first region R1 is 3:2 is set as the lower limit value of L, and a value of L at which a ratio of the cross-sectional area of the second region R2 to the cross-sectional area of the first region R1 is 2:3 is set as the upper limit value of L, ( 1/10^{1/2})·D≤L≤{(3^{1/2})/(20^{1/2})}·D is established, and thus approximately 0.32D≤L≤0.39D is established.

That is, in a case where the outer diameter of the main body part 10 in a natural state is denoted by D and the distance from the axis AX1 of the water-absorbing swelling material 20 having a coil shape to the center line C1 extending in a spiral shape of the wire 21 is denoted by L, it is more preferable that 0.32D≤L≤0.39D is satisfied.

In this way, in a cross section (FIG. 7) perpendicular to the axial direction, the first region R1 and the second region R2 can be filled with the swelled water-absorbing swelling material 20 in a more balanced manner.

It is preferable that L is a value as close to 0.35D as possible.

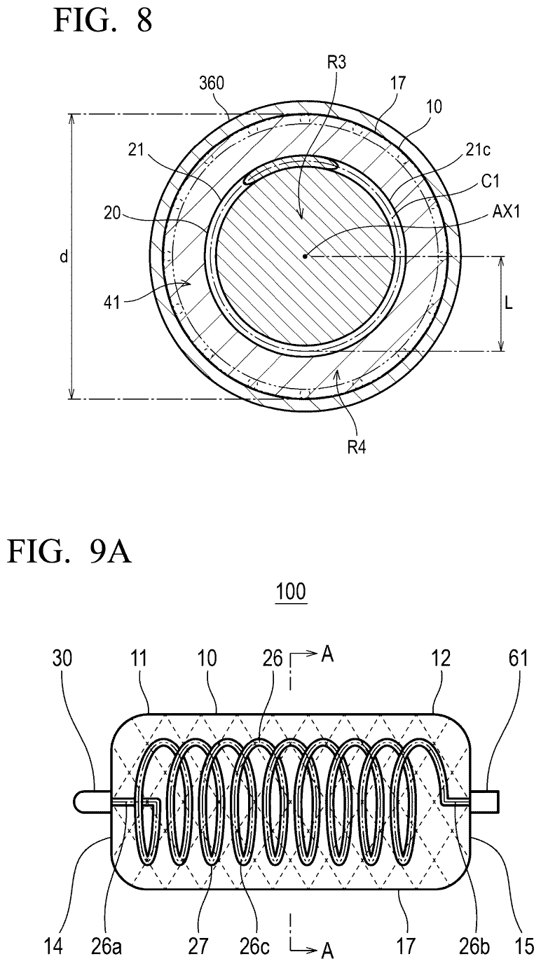

In the above, a preferred relational expression between the outer diameter D of the main body part 10 in a natural state and the length L has been described, but a preferred relational expression between an inner diameter d of the blood vessel in which the intravascular indwelling medical device 100 dwells and the length L will be described below with reference to FIG. 8. In FIG. 8, a third region R3 described below is indicated by hatching upward to the left, and a fourth region R4 described below is indicated by hatching upward to the right. In addition, in FIG. 8, the wire member of the main body part 10 is indicated by a broken line. FIG. 8 shows a cross section taken along line A-A of FIG. 5A.

The intravascular indwelling medical device 100 may have a width in an inner diameter (applicable blood vessel diameter) of the blood vessel 360 for indwelling, for each product model. As an example, in a case where the inner diameter of the blood vessel 360 for indwelling is set to 4 mm or more and 6 mm or less, it is preferable to set the value of L that is appropriate for both of a state in which the intravascular indwelling medical device 100 dwells in the blood vessel 360 having an inner diameter of 6 mm and a state in which the intravascular indwelling medical device 100 dwells in the blood vessel 360 having an inner diameter of 4 mm. Therefore, a numerical range of L is considered to include numerical ranges of both of a case where the intravascular indwelling medical device 100 is designed to be in the best state when dwelling in the blood vessel 360 having an inner diameter of 6 mm and a case where the intravascular indwelling medical device 100 is designed to be in the best state when dwelling in the blood vessel 360 having an inner diameter of 4 mm.

The inner diameter of the blood vessel 360 in which the intravascular indwelling medical device 100 dwells is denoted by d.

It is considered that there is a range of L in which, in a case of being designed such that L=0.25d when dwelling in the blood vessel 360 of 6 mm, 0.25d≤L≤0.41d is satisfied even when dwelling in the blood vessel 360 of 4 mm, in a case of being designed such that L=0.25d when dwelling in the blood vessel 360 of 4 mm, 0.25d≤L≤0.41d is satisfied even when dwelling in the blood vessel 360 of 6 mm, in a case of being designed such that L=0.41d when dwelling in the blood vessel 360 of 6 mm, 0.25d≤L≤0.41d is satisfied even when dwelling in the blood vessel 360 of 4 mm, and in a case of being designed such that L=0.41d when dwelling in the blood vessel 360 of 4 mm, 0.25d≤L≤0.41d is satisfied even when dwelling in the blood vessel 360 of 6 mm.

Then, in a case of being designed such that L=0.25d when dwelling in the blood vessel 360 of 4 mm, that is, in a case where L=1 mm, a proportion of L to the blood vessel 360 of 6 mm is ⅙=approximately 0.17, and the L does not fall within a range of 0.25d≤L≤0.41d. In addition, in a case of being designed such that L=0.41d when dwelling in the blood vessel 360 of 6 mm, that is, in a case where L=2.46 mm, a proportion of L to the blood vessel 360 of 4 mm is 2.46/4=approximately 0.62, and the L does not fall within a range of 0.25d≤L≤0.41d.

Therefore, a preferred relationship between the inner diameter d of the blood vessel in which the intravascular indwelling medical device 100 dwells and L can be, for example, with 0.1d that is slightly smaller than 0.17d as a lower limit of L and 0.7d that is slightly larger than 0.62d as an upper limit of L, 0.1d≤L≤0.7d.

That is, in a case where the inner diameter of the blood vessel in which the intravascular indwelling medical device 100 dwells is denoted by d, by satisfying 0.1d≤L≤0.7d, the value of L can be set to be appropriate for both of a state in which the intravascular indwelling medical device 100 dwells in the blood vessel 360 having an inner diameter of 6 mm and a state in which the intravascular indwelling medical device 100 dwells in the blood vessel 360 having an inner diameter of 4 mm, and the third region R3 and the fourth region R4 can be filled with the swelled water-absorbing swelling material 20 in a well-balanced manner in a cross section (FIG. 8) perpendicular to the axial direction.

Here, the third region R3 is a space inside the water-absorbing swelling material 20 having a coil shape (more precisely, an inner space with respect to the center line C1) in the internal space of the main body part 10. The fourth region R4 is a space outside the water-absorbing swelling material 20 (more precisely, an outer space with respect to the center line C1) in the internal space of the blood vessel 360 in which the intravascular indwelling medical device 100 dwells.

Further, a more preferable relational expression between the inner diameter d of the blood vessel in which the intravascular indwelling medical device 100 dwells and the length L will be described.

It is considered that there is a range of L in which, in a case of being designed such that L=0.32d when dwelling in the blood vessel 360 of 6 mm, 0.32d≤L≤0.39d is satisfied even when dwelling in the blood vessel 360 of 4 mm, in a case of being designed such that L=0.32d when dwelling in the blood vessel 360 of 4 mm, 0.32d≤L≤≤0.39d is satisfied even when dwelling in the blood vessel 360 of 6 mm, in a case of being designed such that L=0.39d when dwelling in the blood vessel 360 of 6 mm, 0.32d≤L≤0.39d is satisfied even when dwelling in the blood vessel 360 of 4 mm, and in a case of being designed such that L=0.39d when dwelling in the blood vessel 360 of 4 mm, 0.32d≤L≤0.39d is satisfied even when dwelling in the blood vessel 360 of 6 mm.

Then, in a case of being designed such that L=0.32d when dwelling in the blood vessel 360 of 6 mm, that is, in a case where L=1.92 mm, a proportion of L to the blood vessel 360 of 4 mm is 1.92/4=approximately 0.48, and the L does not fall within a range of 0.32d≤L≤0.39d. In addition, in a case of being designed such that L=0.32d when dwelling in the blood vessel 360 of 4 mm, that is, in a case where L=1.28 mm, a proportion of L to the blood vessel 360 of 6 mm is 1.28/6=approximately 0.21, and the L does not fall within a range of 0.32d≤L≤0.39d. In addition, in a case of being designed such that L=0.39d when dwelling in the blood vessel 360 of 6 mm, that is, in a case where L=2.34 mm, a proportion of L to the blood vessel 360 of 4 mm is 2.34/4=approximately 0.59, and the L does not fall within a range of 0.32d≤L≤0.39d. In addition, in a case of being designed such that L=0.39d when dwelling in the blood vessel 360 of 4 mm, that is, in a case where L=1.56 mm, a proportion of L to the blood vessel 360 of 6 mm is 1.56/6=approximately 0.26, and the L does not fall within a range of 0.32d≤L≤0.39d.

Therefore, a more preferred relationship between the inner diameter d of the blood vessel in which the intravascular indwelling medical device 100 dwells and L can be, for example, with 0.2d that is slightly smaller than 0.21d as a lower limit of L and 0.6d that is slightly larger than 0.59d as an upper limit of L, 0.2d≤L≤0.6d.

That is, in a case where the inner diameter of the blood vessel in which the intravascular indwelling medical device 100 dwells is denoted by d, by satisfying 0.1d≤L≤0.7d, the value of L can be set to be more appropriate for both of a state in which the intravascular indwelling medical device 100 dwells in the blood vessel 360 having an inner diameter of 6 mm and a state in which the intravascular indwelling medical device 100 dwells in the blood vessel 360 having an inner diameter of 4 mm, and the third region R3 and the fourth region R4 can be filled with the swelled water-absorbing swelling material 20 in a more balanced manner in a cross section (FIG. 8) perpendicular to the axial direction.

Hereinafter, an example of an operation in a case where the intravascular indwelling medical device 100 dwells at a desired site within the blood vessel 360 will be described with reference to FIGS. 4A to 6B. In FKIS. 5A to 5C, the flow of the blood flow is indicated by arrows.

Further, the description will be made from a state in which a guide wire (not shown) for guiding the guide catheter 210 into the blood vessel 360 is inserted in advance.

First, the guide catheter 210 is introduced along the guide wire. More specifically, the guide catheter 210 is externally mounted on the guide wire, and a distal end part 211 of the guide catheter 210 is fed to a desired site within the blood vessel 360 while sliding the guide catheter 210 from the proximal end side to the distal end side along an axial direction of the guide wire. In a case where the distal end part 211 of the guide catheter 210 reaches the desired site, the guide wire is removed.

Subsequently, the pusher wire 220 to which the intravascular indwelling medical device 100 is connected is inserted into the guide catheter 210 and pushed from the proximal end side to the distal end side. As a result, the intravascular indwelling medical device 100 is fed to the distal end part 211 of the guide catheter 210 while sliding the intravascular indwelling medical device 100 from the proximal end side to the distal end side along an inner peripheral surface of the guide catheter 210.

Next, in this state, the intravascular indwelling medical device 100 dwells at the desired site within the blood vessel 360. More specifically, the guide catheter 210 is retracted relative to the intravascular indwelling medical device 100 to remove the guide catheter 210 from the intravascular indwelling medical device 100. As a result, the intravascular indwelling medical device 100 that has been restrained by an inner wall of the guide catheter 210 is restored to the natural state by an elastic restoring force and is expanded in diameter. Further, in a case where the guide catheter 210 is retracted, the retraction of the intravascular indwelling medical device 100 is restricted by the pusher wire 220 that is connected to a proximal end part of the intravascular indwelling medical device 100 (the proximal end part 12 of the main body part 10). As a result, each of the main body part 10 and the water-absorbing swelling material 20 having a coil shape is restored to a shape in a natural state (see FIG. 4B) or a shape close to the natural state by an elastic restoring force thereof. Then, the pusher wire 220 is disconnected from the connection member 61 to remove each of the pusher wire 220 and the guide catheter 210 from the blood vessel 360.

In this way, the intravascular indwelling medical device 100 dwells at a desired site within the blood vessel 360. Further, in the blood vessel 360, the intravascular indwelling medical device 100 dwells in a posture in which, as an example, a distal end side of the intravascular indwelling medical device 100 is disposed on a downstream side of the blood flow and a proximal end side of the intravascular indwelling medical device 100 is disposed on an upstream side of the blood flow. Then, the desired site within the blood vessel 360 is occluded by the swelled water-absorbing swelling material 20 and the thrombi formed inside the main body part 10 and around the main body part 10, and the blood flow is blocked.

More specifically, as shown in FIG. 5A, immediately after the intravascular indwelling medical device 100 dwells, the blood flow passes through the inside of the intravascular indwelling medical device 100 from the proximal end side to the distal end side via the gap 41 between the inner peripheral surface of the main body part 10 and the outer peripheral edge of the water-absorbing swelling material 20 before swelling or the inner cavity of the water-absorbing swelling material 20.

Subsequently, as shown in FIG. 5B, the water-absorbing swelling material 20 absorbs the moisture in the blood and gradually swells with the passage of time. More specifically, as shown in FIGS. 5A and 5B, the entirely of the wire diameter of the water-absorbing swelling material 20 is gradually thickened as compared with before swelling, so that each of the inner diameter of the water-absorbing swelling material 20 and the gap 41 is gradually narrowed. As a result, the growth of the thrombi is promoted in the inner cavity of the water-absorbing swelling material 20 and the gap 41. In addition, a pressure applied to the intravascular indwelling medical device 100 from the blood flow can be gradually increased (the intravascular indwelling medical device 100 can be prevented from suddenly receiving a large pressure from the blood flow), so that a displacement of the intravascular indwelling medical device 100 relative to the blood vessel 360) is suppressed.

As shown in FIG. 5C, the thrombi are formed in the main body part 10 and the water-absorbing swelling material 20, and between the outer peripheral surface 17 of the main body part 10 and the inner wall of the blood vessel 360, and a disposition region of the intravascular indwelling medical device 100 in the blood vessel 360 is occluded. That is, the blood flow from the proximal end side to the distal end side is blocked by the intravascular indwelling medical device 100.

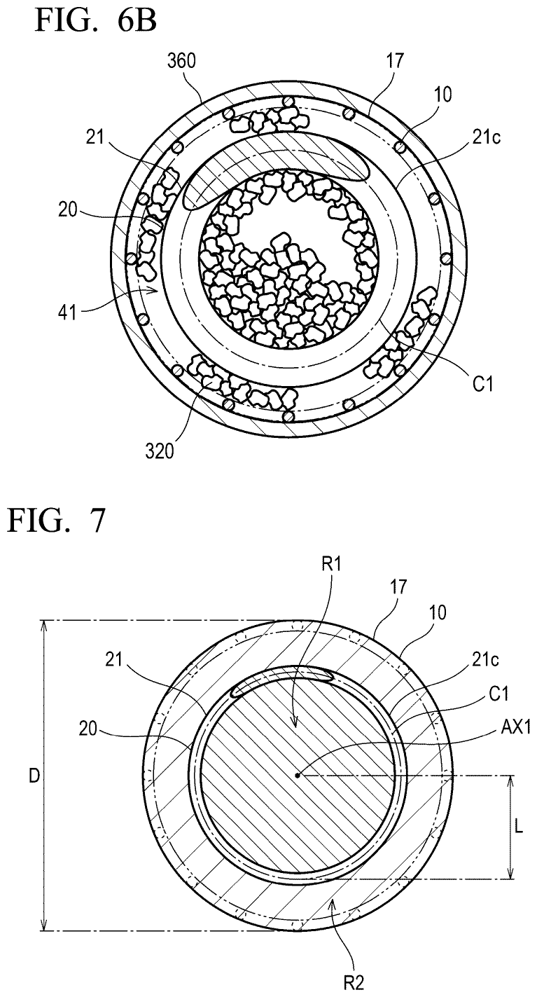

In the example shown in FIG. 5C and FIG. 6C, the thrombi are formed on the proximal end surface 15 of the main body part 10, in the inner cavity of the water-absorbing swelling material 20 and the gap 41, and between the outer peripheral surface 17 of the main body part 10 and the inner wall of the blood vessel 360.

### Modification Example

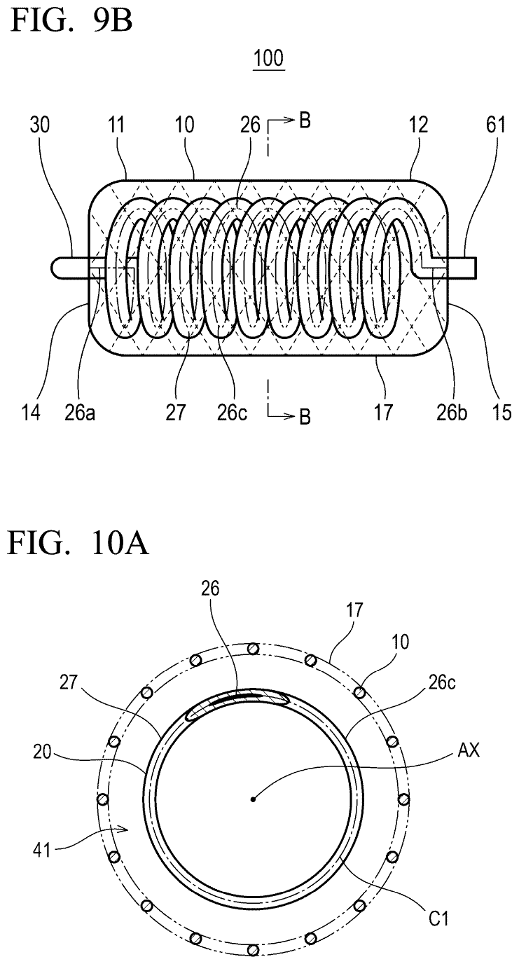

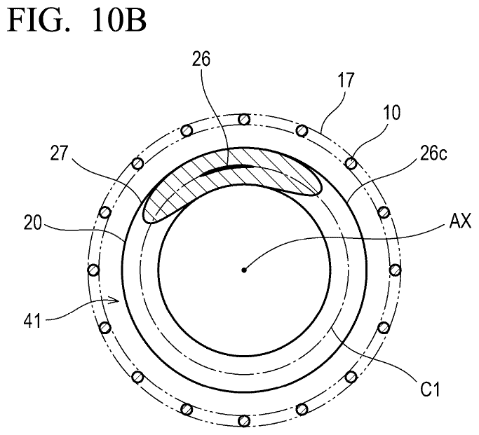

Next, a modification example of the embodiment will be described with reference to FIGS. 9A to 10B. Further, in FIGS. 9A and 9B, the stitches of the main body part 10 are indicated by a broken line for convenience in order to make the internal structure of the main body part 10 easy to see. In addition, in FIGS. 9A and 9B, a metal wire 26 is indicated by a two-dot chain line. In addition, in FIGS. 10A and 10B, the outer peripheral surface and the inner peripheral surface of the main body part 10 are indicated by two-dot chain lines.

As shown in FIGS. 9A to 10B, the present modification example is different from the embodiment described above in that the wire 21 of the water-absorbing swelling material 20 is composed of the metal wire 26 and a water-absorbent polymer 27 that covers a periphery of the metal wire 26 and is the same as the embodiment described above in the other points.

Even with such a configuration, as shown in FIG. 9B and FIG. 10B, the water-absorbent polymer 27 can absorb the moisture in the blood to increase the wire diameter of the water-absorbing swelling material 20. Therefore, the inner diameter of the water-absorbing swelling material 20 after swelling and the gap 41 between the outer peripheral edge of the water-absorbing swelling material 20 after swelling and the inner peripheral surface of the main body part 10 can be made small. Therefore, the growth of the thrombi is promoted in an inner cavity of the water-absorbing swelling material 20 and the gap 41.

In the present modification example as well, the water-absorbing swelling material 20 is formed in, for example, a coil-like wound shape.

More specifically, the metal wire 26 includes, for example, a distal end part 26a, a proximal end part 26b, and an intermediate part 26c that is located between the distal end part 26a and the proximal end part 26b. A distal end of the distal end part 26a is fixed to the distal end part 11 of the main body part 10, and a proximal end of the proximal end part 26b is fixed to the proximal end part 12 of the main body part 10.

Each of the distal end part 26a and the proximal end part 26b extends linearly along the axis AX1 of the water-absorbing swelling material 20 having a coil shape (see FIGS. 9A and 9B).

The intermediate part 26c is wound in a spiral shape. It is preferable that inner diameters and outer diameters of winding parts in the intermediate part 26c are equal to each other. In addition, the intermediate part 26c is formed with an equal pitch as an example.

The intermediate part 26c is disposed, for example, coaxially with the main body part 10. That is, the water-absorbing swelling material 20 is disposed coaxially with the main body part 10.

In addition, in the present embodiment, the water-absorbent polymer 27 covers the entire outer peripheral surface of the metal wire 26.

The intravascular indwelling medical device 100 according to the present invention may be formed of a biodegradable material which has biocompatibility and is decomposed into a harmless compound in a process of normal biological action. Examples of such a biodegradable material include polylactic acid, polyglycolic acid (PGA), collagen or other binding proteins or natural materials, polycaprolactone, hyaluronic acid, adhesive proteins, copolymers, composites, and combinations of these materials as well as combinations with other biodegradable polymers, or biodegradable metallic materials. More specifically, it is preferable that the main body part 10 is formed of, for example, a copolymer of polylactic acid and polyglycolic acid (PLGA), a magnesium alloy, or the like. In addition, it is preferable that the water-absorbing swelling material 20 (wire 21) is formed of, for example, a copolymer of polylactic acid and polyethylene glycol, polysaccharides such as hyaluronic acid and a derivative thereof, polypeptides such as polyaspartic acid, or the like. In addition, it is preferable that the marker member 30 is formed of, for example, a composite of at least any one of the biodegradable material described above and a high-density biocompatible radiopaque filler material such as barium sulfate, bismuth trioxide, tungsten, and tungsten carbide.

Although the embodiment and the modification example have been described with reference to the drawings, these are merely examples of the present invention, and various configurations other than those described above can be adopted.

For example, in the above description, an example is described in which the intravascular indwelling medical device 100 dwells in the blood vessel 360 in a posture in which the distal end side of the intravascular indwelling medical device 100 is disposed on the downstream side of the blood flow and the proximal end side of the intravascular indwelling medical device 100 is disposed on the upstream side of the blood flow. Note that, in the present invention, the posture in a case where the intravascular indwelling medical device 100 dwells is not limited to this example. The intravascular indwelling medical device 100 may dwell, for example, in a posture in which the distal end side of the intravascular indwelling medical device 100 is disposed on the upstream side of the blood flow and the proximal end side of the intravascular indwelling medical device 100 is disposed on the downstream side of the blood flow.

Eyen in such a case, the desired site within the blood vessel 360 can be occluded by the intravascular indwelling medical device 100 and the thrombi formed inside and around the intravascular indwelling medical device 100, and the blood flow can be blocked.

The present embodiment includes the following technical concepts.

(1) An intravascular indwelling medical device that dwells within a blood vessel to occlude the blood vessel, the intravascular indwelling medical device including: a mesh-form main body part; and a water-absorbing swelling material that is disposed within the main body part and swells upon absorbing water.

(2) The intravascular indwelling medical device according to (1), in which the water-absorbing swelling material is fixed to each of a distal end part and a proximal end part of the main body part.

(3) The intravascular indwelling medical device according to (2), in which the water-absorbing swelling material is formed in a shape in which a wire is wound in a coil shape, one end of the wire is fixed to the distal end part of the main body part, and the other end of the wire is fixed to the proximal end part of the main body part.

(4) The intravascular indwelling medical device according to (3), in which the main body part is formed in a hollow cylindrical shape, and in a case where an outer diameter of the main body part in a natural state is denoted by D and a distance from an axis of the water-absorbing swelling material having a coil shape to a center line extending in a spiral shape of the wire is denoted by L, 0.25D<L<0.41D is satisfied.

(5) The intravascular indwelling medical device according to (3) or (4), in which the wire is formed of a water-absorbent polymer.

(6) The intravascular indwelling medical device according to (3) or (4), in which the wire is formed of a metal wire and a water-absorbent polymer that covers a periphery of the metal wire.

(7) The intravascular indwelling medical device according to any one of (2) to (6), in which a connection member for connecting the intravascular indwelling medical device to a pusher wire is provided at the proximal end part of the main body part, and the water-absorbing swelling material is fixed to the proximal end part of the main body part by being fixed to the connection member.

(8) The intravascular indwelling medical device according to any one of (2) to (7), in which a marker member is provided at the distal end part of the main body part, and the water-absorbing swelling material is fixed to the distal end part of the main body part by being fixed to the marker member.

### INDUSTRIAL APPLICABILITY

According to the present invention, there is provided an intravascular indwelling medical device that can occlude a desired site within a blood vessel in a shorter time.

### REFERENCE SIGNS LIST

10: Main body part
11: Distal end part
12: Proximal end part
14: Distal end surface
15: Proximal end surface
17: Outer peripheral surface
20: Water-absorbing swelling material
21: Wire
21a: Distal end part
21b: Proximal end part
21c: Intermediate part
22: One end
23: Other end
26: Metal wire
26a: Distal end part
26b: Proximal end part
26c: Intermediate part
27: Water-absorbent polymer
30: Marker member
41: Gap
61: Connection member
100: Intravascular indwelling medical device
200: Delivery device
210: Guide catheter
211: Distal end part
220: Pusher wire
221: Distal end part
222: Mounting part
320: Thrombi
360: Blood vessel
A: First region
B: Second region
C: Third region
AX1: Axis
D: Outer diameter
d: Inner diameter
L: Distance

1. An intravascular indwelling medical device that dwells within a blood vessel to occlude the blood vessel, the intravascular indwelling medical device comprising:
a mesh-form main body part; and
a water-absorbing swelling material that is disposed within the main body part and swells upon absorbing water.

2. The intravascular indwelling medical device according to claim 1,
wherein the water-absorbing swelling material is fixed to each of a distal end part and a proximal end part of the main body part.

3. The intravascular indwelling medical device according to claim 2,
wherein the water-absorbing swelling material is formed in a shape in which a wire is wound in a coil shape,
one end of the wire is fixed to the distal end part of the main body part, and
the other end of the wire is fixed to the proximal end part of the main body part.

4. The intravascular indwelling medical device according to claim 3,
wherein the main body part is formed in a hollow cylindrical shape, and
in a case where an outer diameter of the main body part in a natural state is denoted by D and a distance from an axis of the water-absorbing swelling material having a coil shape to a center line extending in a spiral shape of the wire is denoted by L, 0.25D<L<0.41D is satisfied.

5. The intravascular indwelling medical device according to claim 3,
wherein the wire is formed of a water-absorbent polymer.

6. The intravascular indwelling medical device according to claim 3,
wherein the wire is formed of a metal wire and a water-absorbent polymer that covers a periphery of the metal wire.

7. The intravascular indwelling medical device according to claim 2,
wherein a connection member for connecting the intravascular indwelling medical device to a pusher wire is provided at the proximal end part of the main body part, and
the water-absorbing swelling material is fixed to the proximal end part of the main body part by being fixed to the connection member.

8. The intravascular indwelling medical device according to claim 2,
wherein a marker member is provided at the distal end part of the main body part, and
the water-absorbing swelling material is fixed to the distal end part of the main body part by being fixed to the marker member.
